(19) United States
(12) Patent Application Publication
(10) Pub. No.: US 2016/0040244 A1
(43) Pub. Date: Feb. 11, 2016

(54) METHOD FOR DETECTING GASTRIC POLYP AND GASTRIC CANCER MARKER GENE OF GASTRIC POLYP AND GASTRIC CANCER-SPECIFIC METHYLATION

(71) Applicant: GENOMICTREE, INC., Daejeon (KR)

(72) Inventors: Sung Whan AN, Daejeon (KR); Tae Jeong OH, Daejeon (KR)

(73) Assignee: Genomictree, Inc., Daejeon (KR)

(21) Appl. No.: 14/438,825

(22) PCT Filed: Sep. 26, 2013
(86) PCT No.: PCT/KR2013/008623

§ 371 Date: Apr. 27, 2015

(30) Foreign Application Priority Data
Nov. 7, 2012 (KR) 10-2012-0125539

(51) Int. Cl. C12Q 1/68

(52) U.S. Cl. CPC C12Q 1/6886; C12Q 2600/154

(57) ABSTRACT

The present invention relates to the novel use of syndecan-2 (SDC2; NM_002998) gene as a gastric polyp- and gastric cancer-specific methylation biomarker, and more particularly, to the use of the syndecan-2 gene as a biomarker that enables gastric polyp and gastric cancer to be diagnosed in an early stage by measuring the methylation level thereof. The present invention has an effect in that the methylation of the CpG island of the gastric polyp- and gastric cancer-specific marker gene can be detected to thereby provide information for diagnosing gastric cancer. The use of the methylation detection method according to the present invention or the diagnostic composition, kit or nucleic acid chip according to the present invention makes it possible to diagnose gastric cancer at an early transformation stage, thus enabling the early diagnosis of gastric cancer. In addition, the method of the present invention enables gastric cancer to be effectively diagnosed in an accurate and rapid manner compared to conventional methods.

### TECHNICAL FIELD

The present invention relates to the novel use of syndecan-2 (SDC2; NM_{—}002998) gene as a gastric polyp- and gastric cancer-specific methylation biomarker, and more particularly, to the use of the syndecan-2 gene as a biomarker that enables gastric polyp and gastric cancer to be diagnosed in an early stage by measuring the methylation level thereof.

### BACKGROUND ART

Even at the present time when medical science has advanced, the 5-year survival rate of cancer patients, particularly solid tumor patients (other than blood cancer patients) is less than 50%, and about ⅔ of all cancer patients are diagnosed at an advanced stage and almost all die within 2 years after cancer diagnosis. Such poor results in cancer therapy are not only the problem of therapeutic methods, but also due to the fact that it not easy to diagnose cancer at an early stage and to accurately diagnose advanced cancer and to carry out the follow-up of cancer patients after cancer therapy.

In current clinical practice, the diagnosis of cancer is confirmed by performing tissue biopsy after history taking, physical examination and clinical assessment, followed by radiographic testing and endoscopy if cancer is suspected. However, the diagnosis of cancer by the existing clinical practices is possible only when the number of cancer cells is more than a billion and the diameter of cancer is more than 1 cm. In this case, the cancer cells already have metastatic ability, and at least half thereof have already metastasized. Meanwhile, tumor markers for monitoring substances that are directly or indirectly produced from cancers are used in cancer screening, but they cause confusion due to limitations in accuracy, since up to about half thereof appear normal even in the presence of cancer, and they often appear positive even in the absence of cancer. Furthermore, the anticancer agents that are mainly used in cancer therapy have the problem that they show an effect only when the volume of cancer is small.

The reason why the diagnosis and treatment of cancer are difficult is that cancer cells are highly complex and variable. Cancer cells grow excessively and continuously, invading surrounding tissue and metastasize to distal organs leading to death. Despite the attack of an immune mechanism or anticancer therapy, cancer cells survive, continually develop, and cell groups that are most suitable for survival selectively propagate. Cancer cells are living bodies with a high degree of viability, which occur by the mutation of a large number of genes. In order that one cell is converted to a cancer cell and developed to a malignant cancer lump that is detectable in clinics, the mutation of a large number of genes must occur. Thus, in order to diagnose and treat cancer at the root, approaches at a gene level are necessary.

Recently, genetic analysis has been actively attempted to diagnose cancer. The simplest typical method is to detect the presence of ABL: BCR fusion genes (the genetic characteristic of leukemia) in blood by PCR. The method has an accuracy rate of more than 95%, and after the diagnosis and therapy of chronic myelocytic leukemia using this simple and easy genetic analysis, this method is being used for the assessment of the result and follow-up study. However, this method has a shortcoming in that it can be applied only to some blood cancers.

Furthermore, another method has been attempted, in which the presence of genes expressed by cancer cells is detected by RT-PCR and blotting, thereby diagnosing cancer cells present in blood cells. However, this method has shortcomings in that it can be applied only to some cancers, including prostate cancer and melanoma, has a high false positive rate. In addition, it is difficult to standardize detection and reading in this method, and its utility is also limited (Kopreski, M. S. et al., Clin. Cancer Res., 5:1961, 1999; Miyashiro, I. et al., Clin. Chem., 47:505, 2001).

Recently, genetic testing that uses a DNA in serum or blood plasma has been actively attempted. This is a method of detecting a cancer-related gene that is isolated from cancer cells and released into blood and present in the form of a free DNA in serum. It is found that the concentration of DNA in serum is increased by a factor of 5-10 times in actual cancer patients as compared to that of normal persons, and such increased DNA is released mostly from cancer cells. The analysis of cancer-specific gene abnormalities, such as the mutation, deletion and functional loss of oncogenes and tumor-suppressor genes, using such DNAs isolated from cancer cells, allows the diagnosis of cancer. In this effort, there has been an active attempt to diagnose lung cancer, head and neck cancer, breast cancer, colon cancer, and liver cancer by examining the promoter methylation of mutated K-Ras oncogenes, p53 tumor-suppressor genes and p16 genes in serum, and the labeling and instability of microsatellite (Chen, X. Q. et al., Clin. Cancer Res., 5:2297, 1999; Esteller, M. et al., Cancer Res., 59:67, 1999; Sanchez-Cespedes, M. et al., Cancer Res., 60:892, 2000; Sozzi, G. et al., Clin. Cancer Res., 5:2689, 1999).

Meanwhile, in samples other than blood, the DNA of cancer cells can also be detected. A method has been attempted in which the presence of cancer cells or oncogenes in sputum or bronchoalveolar lavage of lung cancer patients is detected by a gene or antibody test (Palmisano, W. A. et al., Cancer Res., 60:5954, 2000; Sueoka, E. et al., Cancer Res., 59:1404, 1999). Additionally, other methods of detecting the presence of oncogenes in feces of colon and rectal cancer patients (Ahlquist, D. A. et al., Gastroenterol., 119:1219-27, 2000) and detecting promoter methylation abnormalities in urine and prostate fluid (Goessl, C. et al., Cancer Res., 60:5941, 2000) have been attempted. However, in order to accurately diagnose cancers that cause a large number of gene abnormalities and show various mutations characteristic of each cancer, a method in which a large number of genes are simultaneously analyzed in an accurate and automatic manner is required. However, such a method has not yet been established.

For the accurate diagnosis of cancer, it is important to detect not only a mutated gene but also a mechanism by which the mutation of this gene occurs. Previously, studies were conducted focusing on mutations in a coding sequence, i.e., micro-changes, such as point mutations, deletions and insertions, or macroscopic chromosomal abnormalities. However, in recent years, epigenetic changes were reported to be as important as these mutations, and a typical example of the epigenetic changes is the methylation of promoter CpG islands.

In the genomic DNA of mammal cells, there is the fifth base in addition to A, C, G and T, namely, 5-methylcytosine, in which a methyl group is attached to the fifth carbon of the cytosine ring (5-mC). 5-mC is always attached only to the C of a CG dinucleotide (5′-mCG-3′), which is frequently marked CpG. The C of CpG is mostly methylated by attachment with a methyl group. The methylation of this CpG inhibits a repetitive sequence in genomes, such as Alu or transposon, from being expressed. In addition, this CpG is a site where an epigenetic change in mammalian cells appears most often. The 5-mC of this CpG is naturally deaminated to T, and thus, the CpG in mammal genomes shows only 1% of frequency, which is much lower than a normal frequency (¼×¼=6.25%).

Regions in which CpG are exceptionally integrated are known as CpG islands. The CpG islands refer to sites which are 0.2-3 kb in length, and have a C+G content of more than 50% and a CpG ratio of more than 3.75%. There are about 45,000 CpG islands in the human genome, and they are mostly found in promoter regions regulating the expression of genes. Actually, the CpG islands occur in the promoters of housekeeping genes accounting for about 50% of human genes (Cross, S. et al., Curr. Opin. Gene Develop., 5:309, 1995). Recently, an attempt to examine the promoter methylation of tumor-related genes in blood, sputum, saliva, feces or urine and to use the examined results for the diagnosis and treatment of various cancers, has been actively conducted (Esteller, M. et al., Cancer Res., 59:67, 1999; Sanchez-Cespedez, M. et al., Cancer Res., 60:892, 2000; Ahlquist, D. A. et al., Gastroenterol., 119:1219, 2000). Thus, the present inventors have demonstrated that Syndecan 2 gene can be used specifically to diagnose colon cancer based on relevant studies (KR 10-1142131 B). However, this document does not suggest the use of Syndecan 2 gene for diagnosis of other cancers including gastric cancer. Meanwhile, the present inventors have found that Syndecan 2 gene is not appropriate as a biomarker of various solid cancers such as lung cancer, breast cancer and the like, and thus it acts as only a colon cancer-specific biomarker, rather than a biomarker for diagnosis of general cancers.

Accordingly, the present inventors have made extensive efforts to develop an effective gastric-cancer-specific methylation marker which makes it possible to diagnose cancer and the risk of carcinogenesis at an early stage and predict cancer prognosis. As a result, the present inventors have found that Syndecan 2 (SDC2; NM_{—}002998) gene is methylated specifically in gastric polyps and gastric cancer cells, but not in lung cancer tissue, breast cancer tissue, liver cancer tissue, cervical cancer tissue, and thyroid cancer tissue and that gastric polyps and gastric cancer can be diagnosed at an early stage by measuring the methylation level thereof using the SDC22 gene as a biomarker, thereby completing the present invention.

The above information disclosed in this Background section is only for enhancement of understanding of the background of the present invention, and therefore it may contain information that does not form the prior art that is already known to a person of ordinary skill in the art.

### DISCLOSURE OF INVENTION

### Technical Problem

It is a main object of the present invention to provide a gastric polyp- or gastric cancer-specific methylation biomarker, which is methylated specifically in gastric polyp or gastric cancer, and thus can be effectively used for the diagnosis of gastric polyp or gastric cancer, and the use thereof for providing information for early diagnosis of gastric cancer.

Another object of the present invention is to provide a method for detecting methylation of the SDC2 gene that is a gastric polyp- or gastric cancer-specific methylation marker gene, and a kit and nucleic acid chip for diagnosing gastric polyp or gastric cancer using the SDC2 gene.

### Technical Solution

To achieve the above objects, the present invention provides a kit for diagnosing gastric polyp or gastric cancer, which comprises: a PCR primer pair for amplifying a fragment comprising the CpG island of syndecan-2 (SDC2) gene; and a sequencing primer for pyrosequencing a PCR product amplified by the primer pair.

The present invention also provides a composition for diagnosing gastric polyp or gastric cancer, which contains a substance capable of detecting methylation of the CpG island of syndecan-2 (SDC2) gene.

The present invention also provides a method for detecting gastric polyp or gastric cancer, comprising the steps of:

(a) isolating DNA from a clinical sample; and

(b) measuring the methylation of the CpG island of SDC2 (syndecan-2) gene, which is a gastric polyp or gastric cancer biomarker, in the isolated DNA to detect gastric polyp or gastric cancer.

The present invention also provides a composition for diagnosing gastric polyp or gastric cancer, which contains a substance capable of detecting methylation of the CpG island of the syndecan-2 (SDC2) gene.

The present invention provides a nucleic acid chip for diagnosing gastric polyp or gastric cancer, which has immobilized thereon a probe capable of hybridizing to a fragment comprising the CpG island of syndecan-2 (SDC2) gene under a strict condition.

Other features and embodiments of the present invention will be more apparent from the following detailed descriptions and the appended claims.

### BRIEF DESCRIPTION OF THE DRAWINGS

FIG. 1 is a graph showing the results of measuring the methylation levels of SDC2 biomarker gene, which binds specifically to bisphenol A, in normal gastric tissue, low-grade dysplasia and high-grade dysplasia by pyrosequencing.

FIG. 2A is a graph showing the results of measuring the methylation level of SDC2 biomarker gene in a gastric cancer cell line by pyrosequencing; FIG. 2B is a graph showing the results of measuring the methylation levels of SDC2 biomarker gene in normal gastric tissue, gastric cancer tissue and a normal gastric tissue adjacent to gastric cancer tissue by pyrosequencing; and FIG. 2C shows the results of measuring the sensitivity and specificity of SDC2 biomarker gene for gastric cancer diagnosis by ROC curve analysis.

FIG. 3A shows the results of measuring the methylation levels of SDC2 biomarker gene in the serum DNAs of normal persons and gastric cancer patients by the qMSP method; and FIG. 3B shows the results of measuring the sensitivity and specificity of SDC2 biomarker gene for gastric cancer diagnosis by ROC curve analysis in order to evaluate the ability of the SDC2 biomarker gene to diagnose gastric cancer.

FIG. 4 shows the results of measuring the methylation levels of SDC2 biomarker gene in the cancer tissues and normal tissues of lung cancer, breast cancer, liver cancer, cervical cancer and prostate cancer patients by pyrosequencing.

FIG. 5 shows the results of measuring the methylation levels of SDC2 biomarker gene in the sera of normal persons determined to be normal by gastroscopy and gastric polyp patients, and the results of analyzing the ROC curve.

### BEST MODE FOR CARRYING OUT THE INVENTION

Unless defined otherwise, all technical and scientific terms used herein have the same meaning as commonly understood by one of ordinary skill in the art to which the invention pertains. Generally, the nomenclature used herein are well known and are commonly employed in the art.

The present invention is directed to the use of the CpG island of syndecan-2 (SDC2) gene, which is methylated specifically in gastric polyp or gastric cancer, as a biomarker. Accordingly, in one aspect, the present invention is directed to a composition for diagnosing gastric polyp or gastric cancer, which contains a substance capable of detecting methylation of the CpG island of the syndecan-2 (SDC2) gene.

In the present invention, the CpG island may be located in the promoter region of the SDC2 gene. In addition, it may also be located in the regions upstream or downstream of the promoter, such as an intron, exon or enhancer region.

In the present invention, the CpG island is preferably located in the promoter region, 5′-untranslated region, first exon and first intron of the SDC2 gene. Specifically, the CpG island may be located between −0.5 kb and +1.5 kb from the transcription initiation point of the SDC2 (syndecan-2) gene. More specifically, the CpG island may be located in a region represented by SEQ ID NO: 1.

The gastric polyp- or gastric cancer-specific methylation marker genes of the present invention can be used for gastric cancer screening, risk-assessment, prognosis, disease identification, the diagnosis of disease stages, and the selection of therapeutic targets. Particularly, the SDC2 gene that is a biomarker gene according to the present invention showed a high level and frequency of positive methylation in gastric polyp that is a precancerous lesion of gastric cancer, suggesting that the SDC2 gene is useful as a biomarker for diagnosis of gastric polyp and early diagnosis of gastric cancer.

In the present invention, the substance capable of detecting methylation of the CpG island may be any one selected from the group consisting of a primer pair capable of amplifying a fragment comprising the methylated CpG island, a probe capable of hybridizing to the methylated CpG island, a methylation-specific binding protein or a methylation-specific binding antibody which is capable of binding to the methylated CpG island, a sequencing primer, a sequencing-by-synthesis primer, and a sequencing-by-ligation primer.

The identification of genes that are methylated in gastric cancer and abnormalities at various stages of gastric cancer makes it possible to diagnose gastric cancer at an early stage in an accurate and effective manner and allows methylation profiling of multiple genes and the identification of new targets for therapeutic intervention. Furthermore, the methylation data according to the present invention may be combined with other non-methylation related biomarker detection methods to obtain a more accurate system for gastric cancer diagnosis.

According to the method of the present invention, the progression of gastric cancer at various stages or phases can be diagnosed by determining the methylation stage of one or more nucleic acid biomarkers obtained from a sample. By comparing the methylation stage of a nucleic acid isolated from a sample at each stage of gastric cancer with the methylation stage of one or more nucleic acids isolated from a sample in which there is no cell proliferative disorder of gastric tissue, a specific stage of gastric cancer in the sample can be detected. Herein, the methylation stage may be hypermethylation.

In one embodiment of the present invention, nucleic acid may be methylated in the regulatory region of a gene. In another embodiment, a gene which is involved in cell transformation can be diagnosed by detecting methylation outside of the regulatory region of the gene, because methylation proceeds inwards from the outside of the gene.

One example of the kit of the present invention includes: a carrier means compartmentalized to receive a sample therein; and one or more containers comprising a first container containing a reagent which sensitively cleaves unmethylated cytosine, a second container containing primers for amplification of a CpG-containing nucleic acid, and a third container containing a means to detect the presence of cleaved or uncleaned nucleic acid. Primers contemplated for use in accordance with the invention include the sequences set forth in SEQ ID NOS: 2, 3, 5 and 6, and any functional combination and fragments thereof. The functional combination or fragment is used as a primer to detect whether methylation has occurred on the region of the genome sought to be detected.

In addition, according to the present invention, a cellular proliferative disorder (dysplasia) of gastric tissue in a sample can be diagnosed by detecting methylation of the SDC2 (syndecan-2) gene using a kit or nucleic acid chip.

Accordingly, in another aspect, the present invention is directed to a modified nucleic acid for diagnosis of gastric polyp or gastric cancer, derived from a SDC2 (syndecan-2) gene fragment set forth in SEQ ID NO: 1, in which the modified nucleic acid is obtained by modifying the SDC2 gene fragment so that at least one cytosine residue in the SDC2 gene fragment will be modified to uracil or a nucleotide other than cytosine in a hybridization process.

In the present invention, the modified nucleic acid may comprise a sequence set forth in SEQ ID NO: 23.

In another aspect, the present invention is directed to a kit for diagnosing gastric polyp or gastric cancer, which comprises: a PCR primer pair for amplifying a fragment comprising the CpG island of syndecan-2 (SDC2) gene; and a sequencing primer for pyrosequencing a PCR product amplified by the primer pair.

The present invention is also directed to a nucleic acid chip for diagnosing gastric polyp or gastric cancer, which has immobilized thereon a probe capable of hybridizing to a fragment comprising the CpG island of syndecan-2 (SDC2) gene under a strict condition.

The use of the diagnostic kit or nucleic acid chip allows for the detection of a cellular proliferative disorder (dysplasia) of gastric tissue in a sample. The detection method comprises determining the methylation state of at least one nucleic acid isolated from a sample, and the methylation state of the at least one nucleic acid may be compared with the methylation state of a nucleic acid isolated from a sample in which there is no cellular proliferative disorder (dysplasia) of gastric tissue.

In yet another embodiment of the present invention, cells that are likely to form gastric cancer can be diagnosed at an early stage using the methylation marker genes. When genes confirmed to be methylated in cancer cells are methylated in cells that appear normal clinically or morphologically, this indicates that the normally appearing cells progress to cancer. Thus, gastric cancer can be diagnosed at an early stage by detecting the methylation of gastric cancer-specific genes in cells that appear normally. Particularly, in an example of the present invention, it was found that the SDC2 (syndecan-2) gene can be used for diagnosis of gastric polyp that is a precancerous lesion of gastric cancer

The present invention enables, cells that are likely to form gastric cancer, to be diagnosed at an early stage using the methylation marker genes. When genes confirmed to be methylated in cancer cells are methylated in cells that appear normal clinically or morphologically, this indicates that the normally appearing cells progress to cancer. Thus, gastric cancer can be diagnosed at an early stage by detecting the methylation of gastric cancer-specific genes in cells that appear normally.

The use of the methylation marker gene of the present invention allows for detection of a cellular proliferative disorder (dysplasia) of gastric tissue in a sample. The detection method comprises bringing a sample comprising at least one nucleic acid isolated from a subject into contact with at least one agent capable of determining the methylation state of the nucleic acid. The method comprises detecting the methylation of at least one region in at least one nucleic acid, wherein the methylation of the nucleic acid differs from the methylation state of the same region of a nucleic acid present in a sample in which there is no abnormal growth (dysplastic progression) of gastric cells.

In yet another embodiment of the present invention, the likelihood of progression of tissue to gastric cancer can be evaluated by examining the methylation frequency of a gene which is specifically methylated in gastric cancer, and determining the methylation frequency of tissue that is likely to progress to gastric cancer.

Therefore, in still another aspect, the present invention is directed to a method for detecting gastric polyp or gastric cancer, comprising the steps of:

(a) isolating DNA from a clinical sample; and

(b) measuring the methylation of the CpG island of SDC2 (syndecan-2) gene, which is a gastric polyp or gastric cancer biomarker, in the isolated DNA to detect gastric polyp or gastric cancer.

In the present invention, step (b) may be performed by measuring the methylation of any one of the promoter, 5′-untranslated region (UTR), intron and exon regions of the gene. Preferably, the methylation of the CpG island in a region of the SDC2 gene, which has a nucleotide sequence of SEQ ID NO: 1 may be measured.

In the present invention, step (b) may be performed by a method selected from the group consisting of PCR, methylation-specific PCR, real-time methylation-specific PCR, PCR assay using a methylation DNA-specific binding protein, quantitative PCR, DNA chip-based assay, pyrosequencing, and bisulfate sequencing. In addition, the clinical sample may be selected from the group consisting of a tissue, cell, blood, blood plasma, feces, and urine from a patient suspected of cancer or a subject to be diagnosed, but is not limited thereto.

In one embodiment of the present invention, the method for detecting the methylation of a gene may comprise: (a) preparing a clinical sample containing DNA; (b) isolating DNA from the clinical sample; (c) amplifying the isolated DNA using primers capable of amplifying a fragment comprising the CpG island of the promoter or intron of an SDC2 gene; and (d) determining whether the intron was methylated based on whether the DNA was amplified in step (c).

In another embodiment of the present invention, a cellular proliferative disorder (dysplasia) of gastric tissue in a sample can be diagnosed by detecting the methylation state of the SDC2 (NM_{—}002998, Syndecan 2) gene using a kit.

Thus, in still another aspect, the present invention is directed to a kit for diagnosing gastric polyp or gastric cancer, which contains: a PCR primer pair for amplifying a fragment comprising either the CpG island of the SDC2 (NM_{—}002998, Syndecan 2) gene; and a sequencing primer for pyrosequencing a PCR product amplified by the primer pair:

In the present invention, the PCR primer pair may be a primer pair set forth in SEQ ID NO: 2 and 3, or a primer pair set forth in SEQ ID NOS: 5 and 6.

In the present invention, the sequencing primer may be a primer set forth in SEQ ID NO: 4 or 7.

In another embodiment of the present invention, cellular proliferative disorder (dysplasia) of gastric tissue cells in a sample can be diagnosed by detecting the methylation state of the SDC2 (NM_{—}002998, Syndecan 2) gene using a nucleic acid chip.

In still another aspect, the present invention is directed to a nucleic acid chip for diagnosing gastric polyp or gastric cancer, which has immobilized thereon a probe capable of hybridizing to a fragment comprising the CpG island of syndecan-2 (SDC2) gene under a strict condition.

In the present invention, the probe may be selected from the group consisting of the base sequences set forth in SEQ ID NOS: 8 to 19 and specific examples thereof are as follows:

SDC2
1)
(SEQ ID NO: 8)
5′-cggagctgcc aatcggcgtg taatcctgta-3′
2)
(SEQ ID NO: 9)
5′-ctgccgtagc tccctttcaa gccagcgaat ttattcctta
aaaccagaaa-3′
3)
(SEQ ID NO: 10)
5′-gcacgggaaa ggagtccgcg gaggagcaaa accacagcag
agcaagaaga-3′
4)
(SEQ ID NO: 11)
5′-gcagccttcc cggagcacca actccgtgtc gggagtgcag
aaaccaacaa gtgagagggc-3′
5)
(SEQ ID NO: 12)
5′-cccgagcccg agtccccgag cctgagccgc aatcgctgcg
gtactctgct-3′
6)
(SEQ ID NO: 13)
5′-cttggtggcc tgcgtgtcgg cggagtcggt gagtgggcca-3′
Modified nucleic acid sequence probe
1′)
(SEQ ID NO: 14)
5′-cggagttgtt aatcggcgtg taattttgta-3′
2′)
(SEQ ID NO: 15)
5′-ttgtcgtagt ttttttttaa gttagcgaat ttatttttta
aaattagaaa-3′
3′)
(SEQ ID NO: 16)
5′-gtacgggaaa ggagttcgcg gaggagtaaa attatagtag
agtaagaaga-3′
4′)
(SEQ ID NO: 17)
5′-gtagtttttt cggagtatta atttcgtgtc gggagtgtag
aaattaataa gtgagagggt-3′
5′)
(SEQ ID NO: 18)
5′-ttcgagttcg agttttcgag tttgagtcgt aatcgttgcg
gtattttgtt-3′
6′)
(SEQ ID NO: 19)
5′-tttggtggtt tgcgtgtcgg cggagtcggt gagtgggtta-3′

The use of the diagnostic kit or nucleic acid chip of the present invention makes it possible to determine the abnormal growth (dysplastic progression) of gastric tissue cells in a sample. The method comprises determining the methylation state of at least one nucleic acid isolated from a sample, wherein the methylation state of the at least one nucleic acid is compared with the methylation stage of a nucleic acid isolated from a sample in which there is no abnormal growth (dysplastic progression) of gastric cells.

In another embodiment of the present invention, transformed gastric cancer cells can be detected by examining the methylation of the marker gene using said kit or nucleic acid chip.

In still another embodiment of the present invention, gastric cancer can be diagnosed by examining the methylation of the marker gene using said kit or nucleic acid chip.

In yet another embodiment of the present invention, the likelihood of progression to gastric cancer can be diagnosed by examining the methylation of the marker gene in a sample showing a normal phenotype using said kit or nucleic acid chip. The sample that is used in the present invention may be solid or liquid tissue, cells, feces, urine, serum, or blood plasma.

Major terms which are used herein are defined as follows.

As used herein, the term “cell transformation” refers to the change in characteristics of a cell from one form to another form such as from normal to abnormal, non-tumorous to tumorous, undifferentiated to differentiated, stem cell to non-stem cell. In addition, the transformation can be recognized by the morphology, phenotype, biochemical characteristics and the like of a cell.

As used herein, the term “early detection” of cancer refers to discovering the likelihood of cancer prior to metastasis, and preferably before observation of a morphological change in a tissue or cell. Furthermore, the term “early detection” of cell transformation refers to the high probability of a cell to undergo transformation in its early stages before the cell is morphologically designated as being transformed.

As used herein, the term “hypermethylation” refers to the methylation of a CpG island.

As used herein, the term “sample” or “clinical sample” is referred to in its broadest sense, and includes any biological sample obtained from an individual, body fluid, a cell line, a tissue culture, depending on the type of assay that is to be performed. Methods for obtaining tissue biopsies and body fluids from mammals are well known in the art. A gastric tissue biopsy is a preferred source.

Use of Cancer Cells for Comparison Between Gastric Cancer Biomarker and Normal Cells

In the present invention, “normal” cells refer to those that do not show any abnormal morphological or cytological changes. “Tumor” cells are cancer cells. “Non-tumor” cells are those cells that are part of the diseased tissue but are not considered to be the tumor portion.

In one aspect, the present invention is based on the discovery of the relationship between gastric cancer and the hypermethylation of SDC2 (NM_{—}002998, Syndecan 2) gene.

In another embodiment of the present invention, a cellular proliferative disorder of gastric tissue cell can be diagnosed at an early stage by determining the methylation stage of at least one nucleic acid from a subject using the kit or nucleic acid chip of the present invention. Herein, the methylation stage of the at least one nucleic acid may be compared with the methylation state of at least one nucleic acid isolated from a subject not having a cellular proliferative disorder of gastric tissue. The nucleic acid is preferably a CpG-containing nucleic acid such as a CpG island.

In another embodiment of the present invention, a cellular proliferative disorder of gastric tissue can be diagnosed by determining the methylation of at least one nucleic acid from a subject using the kit or nucleic acid chip of the present invention. Herein, the nucleic acid may be SDC2 (NM_{—}002998, Syndecan 2). In this embodiment, the methylation of the at least one nucleic acid may be compared with the methylation state of at least one nucleic acid isolated from a subject having no predisposition to a cellular proliferative disorder of gastric tissue.

As used herein, “predisposition” refers to the property of being susceptible to a cellular proliferative disorder. A subject having a predisposition to a cellular proliferative disorder has no cellular proliferative disorder, but is a subject having an increased likelihood of having a cellular proliferative disorder.

In another aspect, the present invention provides a method for diagnosing a cellular proliferative disorder of gastric tissue, the method comprising brining a sample comprising a nucleic acid into contact with an agent capable of determining the methylation state of the sample, and determining the methylation of at least one region of the at least one nucleic acid. Herein, the methylation of the at least one region in the at least one nucleic acid differs from the methylation stage of the same region in a nucleic acid present in a subject in which there is no abnormal growth of cells.

The method of the present invention comprises a step of determining the methylation of at least one region of at least one nucleic acid isolated from a subject.

The term “nucleic acid” or “nucleic acid sequence” as used herein refers to an oligonucleotide, nucleotide or polynucleotide, or fragments thereof, or single-stranded or double-stranded DNA or RNA of genomic or synthetic origin, sense- or antisense-strand DNA or RNA of genomic or synthetic origin, peptide nucleic acid (PNA), or any DNA-like or RNA-like material of natural or synthetic origin. It will apparent to those of skill in the art that, when the nucleic acid is RNA, the deoxynucleotides A, G, C, and T are replaced by the ribonucleotides A, G, C, and U, respectively.

Any nucleic acid may be used in the present invention, given the presence of differently methylated CpG islands can be detected therein. The CpG island is a CpG-rich region in a nucleic acid sequence.

Methylation

In the present invention, any nucleic acid sample, in purified or non-purified form, can be used, provided it contains or is suspected of containing a nucleic acid sequence containing a target locus (e.g., CpG-containing nucleic acid). One nucleic acid region capable of being differentially methylated is a CpG island, a sequence of nucleic acid with an increased density relative to other nucleic acid regions of the dinucleotide CpG. The CpG doublet occurs in vertebrate DNA at only about 20% of the frequency that would be expected from the proportion of G*C base pairs. In certain regions, the density of CpG doublets reaches the predicted value; it is increased by ten-fold relative to the rest of the genome. CpG islands have an average G*C content of about 60%, compared with the 40% average in bulk DNA. The islands take the form of stretches of DNA typically about one to two kilobases long. There are about 45,000 islands in the human genome.

In many genes, the CpG islands begin just upstream of a promoter and extend downstream into the transcribed region. Methylation of a CpG island at a promoter usually suppresses expression of the gene. The islands can also surround the 5′ region of the coding region of the gene as well as the 3′ region of the coding region. Thus, CpG islands can be found in multiple regions of a nucleic acid sequence including upstream of coding sequences in a regulatory region including a promoter region, in the coding regions (e.g., exons), downstream of coding regions in, for example, enhancer regions, and in introns.

Typically, the CpG-containing nucleic acid is DNA. However, the inventive method may employ, for example, samples that contain DNA, or DNA and RNA containing mRNA, wherein DNA or RNA may be single-stranded or double-stranded, or a DNA-RNA hybrid may be included in the sample.

A mixture of nucleic acids may also be used. The specific nucleic acid sequence to be detected may be a fraction of a larger molecule or can be present initially as a discrete molecule, so that the specific sequence constitutes the entire nucleic acid. It is not necessary that the sequence to be studied be present initially in a pure form; the nucleic acid may be a minor fraction of a complex mixture, such as contained in whole human DNA. Nucleic acids contained in a sample used for detection of methylated CpG islands may be extracted by a variety of techniques such as that described by Sambrook, et al. (Molecular Cloning: A Laboratory Manual, Cold Spring Harbor, N. Y., 1989).

Nucleic acids isolated from a subject are obtained in a biological sample from the subject. If it is desired to detect gastric cancer or stages of gastric cancer progression, the nucleic acid may be isolated from gastric tissue by scraping or biopsy. Such samples may be obtained by various medical procedures known to those of skill in the art.

In one aspect of the invention, the state of methylation in nucleic acids of the sample obtained from a subject is hypermethylation compared with the same regions of the nucleic acid in a subject not having a cellular proliferative disorder of gastric tissue. Hypermethylation as used herein refers to the presence of methylated alleles in one or more nucleic acids. Nucleic acids from a subject not having a cellular proliferative disorder of gastric tissue contain no detectable methylated alleles when the same nucleic acids are examined.

Individual Genes and Panel

It is understood that the present invention may be practiced using each gene separately as a diagnostic or prognostic marker or a few marker genes combined into a panel display format so that several marker genes may be detected for overall pattern or listing of genes that are methylated to increase reliability and efficiency. Furthermore, any of the genes identified in the present invention may be used individually or as a set of genes in any combination with any of the other genes that are recited herein. Alternatively, genes may be ranked according to their importance and weighted and together with the number of genes that are methylated, a level of likelihood of developing cancer may be assigned. Such algorithms are within the scope of the present invention.

Method for Detection of Methylation

The present invention is directed to a method for diagnosing gastric polyp or gastric cancer, which comprises treating genomic DNA, isolated from a clinical sample, with bisulfite to convert the SDC2 CpG site, and detecting the methylation of the converted SDC2 CpG site using at least one synthetic oligonucleotide which is complementary thereto or is capable of hybridizing thereto.

Methylation-Specific PCR

When genomic DNA is treated with bisulfite, cytosine in the 5′-CpG′-3 region remains intact, if it was methylated, but the cytosine changes to uracil, if it was unmethylated. Accordingly, based on the base sequence converted after bisulfite treatment, PCR primer sets corresponding to a region having the 5′-CpG-3′ base sequence are constructed. Herein, the constructed primer sets are two kinds of primer sets: a primer set corresponding to the methylated base sequence, and a primer set corresponding to the unmethylated base sequence. When genomic DNA is converted with bisulfite and then amplified by PCR using the above two kinds of primer sets, the PCR product is detected in the PCR mixture employing the primers corresponding to the methylated base sequence, if the genomic DNA was methylated, but the genomic DNA is detected in the PCR mixture employing the primers corresponding to the unmethylated, if the genomic DNA was unmethylated. This methylation can be quantitatively analyzed by agarose gel electrophoresis.

Real-Time Methylation Specific PCR

Real-time methylation-specific PCR is a real-time measurement method modified from the methylation-specific PCR method and comprises treating genomic DNA with bisulfite, designing PCR primers corresponding to the methylated base sequence, and performing real-time PCR using the primers. Methods of detecting the methylation of the genomic DNA include two methods: a method of detection using a TanMan probe complementary to the amplified base sequence; and a method of detection using Sybergreen. Thus, the real-time methylation-specific PCR allows selective quantitative analysis of methylated DNA. Herein, a standard curve is plotted using an in vitro methylated DNA sample, and a gene containing no 5′-CpG-3′ sequence in the base sequence is also amplified as a negative control group for standardization to quantitatively analyze the degree of methylation.

Pyrosequencing

The pyrosequencing method is a quantitative real-time sequencing method modified from the bisulfite sequencing method. Similarly to bisulfite sequencing, genomic DNA is converted by bisulfite treatment, and then, PCR primers corresponding to a region containing no 5′-CpG-3′ base sequence are constructed. Specifically, the genomic DNA is treated with bisulfite, amplified using the PCR primers, and then subjected to real-time base sequence analysis using a sequencing primer. The degree of methylation is expressed as a methylation index by analyzing the amounts of cytosine and thymine in the 5′-CpG-3′ region.

PCR Using Methylated DNA-Specific Binding Protein, Quantitative PCR, and DNA Chip Assay

When a protein binding specifically only to methylated DNA is mixed with DNA, the protein binds specifically only to the methylated DNA. Thus, either PCR using a methylation-specific binding protein or a DNA chip assay allows selective isolation of only methylated DNA. Genomic DNA is mixed with a methylation-specific binding protein, and then only methylated DNA was selectively isolated. The isolated DNA is amplified using PCR primers corresponding to the promoter region, and then methylation of the DNA is measured by agarose gel electrophoresis.

In addition, methylation of DNA can also be measured by a quantitative PCR method, and methylated DNA isolated with a methylated DNA-specific binding protein can be labeled with a fluorescent probe and hybridized to a DNA chip containing complementary probes, thereby measuring methylation of the DNA. Herein, the methylated DNA-specific binding protein may be, but not limited to, MBD2bt (truncated methyl CpG binding domain protein 2).

Detection of Differential Methylation-Methylation-Sensitive Restriction Endonuclease

Detection of differential methylation can be accomplished by bringing a nucleic acid sample into contact with a methylation-sensitive restriction endonuclease that cleaves only unmethylated CpG sites.

In a separate reaction, the sample is further brought into contact with an isoschizomer of the methylation-sensitive restriction enzyme that cleaves both methylated and unmethylated CpG-sites, thereby cleaving the methylated nucleic acid.

Specific primers are added to the nucleic acid sample, and the nucleic acid is amplified by any conventional method. The presence of an amplified product in the sample treated with the methylation-sensitive restriction enzyme but absence of an amplified product in the sample treated with the isoschizomer of the methylation-sensitive restriction enzyme indicates that methylation has occurred at the nucleic acid region assayed. However, the absence of an amplified product in the sample treated with the methylation-sensitive restriction enzyme together with the absence of an amplified product in the sample treated with the isoschizomer of the methylation-sensitive restriction enzyme indicates that no methylation has occurred at the nucleic acid region assayed.

As used herein, the term “methylation-sensitive restriction enzyme” refers to a restriction enzyme (e.g., Smal) that includes CG as part of its recognition site and has activity when the C is methylated as compared to when the C is not methylated. Non-limiting examples of methylation-sensitive restriction enzymes include MspI, HpaII, BssHII, BstUI and NotI. Such enzymes can be used alone or in combination. Examples of other methylation-sensitive restriction enzymes include, but are not limited to SacII and EagI.

The isoschizomer of the methylation-sensitive restriction enzyme is a restriction enzyme that recognizes the same recognition site as the methylation-sensitive restriction enzyme but cleaves both methylated and unmethylated CGs. An example thereof includes MspI.

Primers of the present invention are designed to be “substantially” complementary to each strand of the locus to be amplified and include the appropriate G or C nucleotides as discussed above. This means that the primers must be sufficiently complementary to hybridize with their respective strands under polymerization reaction conditions. Primers of the present invention are used in the amplification process, which is an enzymatic chain reaction (e.g., PCR) in which that a target locus exponentially increases through a number of reaction steps. Typically, one primer is homologous with the negative (−) strand of the locus (antisense primer), and the other primer is homologous with the positive (+) strand (sense primer). After the primers have been annealed to denatured nucleic acid, the nucleic acid chain is extended by an enzyme such as DNA Polymerase I (Klenow), and reactants such as nucleotides, and, as a result, + and − strands containing the target locus sequence are newly synthesized. When the newly synthesized target locus is used as a template and subjected to repeated cycles of denaturing, primer annealing, and extension, exponential synthesis of the target locus sequence occurs. The resulting reaction product is a discrete nucleic acid duplex with termini corresponding to the ends of specific primers employed.

The amplification reaction is PCR which is commonly used in the art. However, alternative methods such as real-time PCR or linear amplification using isothermal enzyme may also be used. In addition, multiplex amplification reactions may also be used.

Bisulfite Sequencing Method

Another method for detecting a methylated CpG-containing nucleic acid comprises bringing a nucleic acid-containing sample into contact with a reagent that modifies unmethylated cytosine, and amplifying the CpG-containing nucleic acid in the sample using methylation-independent oligonucleotide primers. Herein, the oligonucleotide primers amplify nucleic acid without distinguishing between modified methylated nucleic acid and unmethylated nucleic acid. The amplified product is sequenced by the Sanger method using a sequencing primer or sequenced by a next-generation sequencing method described in relation to bisulfite sequencing for detection of methylated nucleic acid. Herein, the next-generation sequencing method may be performed by a sequencing-by-synthesis or sequencing-by-ligation technique. This method is characterized in that, instead of preparing bacterial clones, a single DNA fragment is isolated spatially, amplified in situ (clonal amplification), and sequenced. Herein, hundreds of thousands of fragments are read out at the same time, and for this reason, the method is also called “massively parallel sequencing method”.

Typically, the sequencing-by-synthesis method is used in which signals are obtained while mono- or di-nucleotides are sequentially added. Examples of this method include pyrosequencing, ion torrent, and Solexa methods.

NGS systems based on sequencing-by-synthesis include 454 platform (Roche), HiSeq platform (Illumina), Ion PGM platform (Life Technology), and PacBio platform (Pacific BioSciences). The 454 and Ion PGM platforms use emersion PCR for clonal amplification, and the HiSeq platform uses Bridge amplification. In the sequencing-by-synthesis method, sequencing is performed by detecting phosphate, hydrogen ion, or fluorescence, which is generated when DNA is synthesized by sequentially adding single nucleotides. In the process of detecting sequences, the 454 platform is based on pyrosequencing, and the Ion PGM platform is based on the detection of hydrogen ion. The HiSeq and PacBio platforms perform sequencing by detecting fluorescence.

The sequencing-by-ligation method is a sequencing technique that uses DNA ligase, and is performed to identify a nucleotide present at a specific position of a DNA nucleotide sequence. Unlike most sequencing techniques that use polymerase, the sequencing-by-ligation method is characterized in that polymerase is not used and DNA ligase does not ligate mismatched sequences. The SOLiD system corresponds to this method. In this technique, two nucleotides are read at every step of the sequencing process. The reading is individually repeated five times by primer reset, and thus each nucleotide is read twice, making the data highly accurate.

In the sequencing-by-ligation method, among dinucleotide primer sets made of 16 combinations, dinucleotide primers corresponding to the nucleotide sequence of interest are sequentially ligated, and a combination of the ligations is finally analyzed to determine the nucleotide sequence of the DNA of interest.

Sequencing, Sequencing-by-Synthesis or Sequencing-by-Ligation that Use Methylated DNA-Specific Binding Protein or Antibody

In a sequencing or next-generation sequencing method that uses a methylated DNA-specific binding protein or antibody, a protein or antibody that binds specifically to methylated DNA is mixed with DNA, and then it binds specifically to methylated DNA. Thus, only methylated DNA can be selectively isolated. In the present invention, genomic DNA was mixed with a methylated DNA-specific binding protein, and then only methylated DNA was selectively isolated. The isolated DNA was amplified using PCR primers, and then whether the DNA was methylated was measured by the Sanger method or the sequencing-by-synthesis or sequencing-by-ligation method.

Herein, the next-generation sequencing method may be performed by the sequencing-by-synthesis or sequencing-by-ligation method. In addition, the methylated DNA-specific binding protein may be MBD2bt, but is not limited thereto, and the antibody may be 5′-methyl-cytosine, but is not limited thereto.

Kit

The present invention provides a kit useful for the detection of a cellular proliferative disorder in a subject. The kit of the present invention comprises a carrier means compartmentalized to receive a sample therein, one or more containers comprising a second container containing PCR primers for amplification of a 5′-CpG-3′ base sequence, and a third container containing a sequencing primer for pyrosequencing an amplified PCR product.

Carrier means are suited for containing one or more containers such as vials, tubes, and the like, each of the containers comprising one of the separate elements to be used in the method. In view of the description provided herein of the inventive method, those of skill in the art can readily determine the apportionment of the necessary reagents among the containers.

Substrates

After the target nucleic acid region has been amplified, the nucleic acid amplification product can be hybridized to a known gene probe attached to a solid support (substrate) to detect the presence of the nucleic acid sequence.

As used herein, the term “substrate”, when used in reference to a substance, structure, surface or material, means a composition comprising a nonbiological, synthetic, nonliving, planar or round surface that is not heretofore known to comprise a specific binding, hybridization or catalytic recognition site or a plurality of different recognition sites or a number of different recognition sites which exceeds the number of different molecular species comprising the surface, structure or material. Examples of the substrate include, but are not limited to, semiconductors, synthetic (organic) metals, synthetic semiconductors, insulators and dopants; metals, alloys, elements, compounds and minerals; synthetic, cleaved, etched, lithographed, printed, machined and microfabricated slides, devices, structures and surfaces; industrial polymers, plastics, membranes silicon, silicates, glass, metals and ceramics; and wood, paper, cardboard, cotton, wool, cloth, woven and nonwoven fibers, materials and fabrics; and amphibious surfaces.

It is known in the art that several types of membranes have adhesion to nucleic acid sequences. Specific non-limiting examples of these membranes include nitrocellulose or other membranes used for detection of gene expression such as polyvinylchloride, diazotized paper and other commercially available membranes such as GENESCREEN™, ZETAPROBE™ (Biorad) and NYTRAN™. Beads, glass, wafer and metal substrates are also included. Methods for attaching nucleic acids to these objects are well known in the art. Alternatively, screening can be done in a liquid phase.

Hybridization Conditions

In nucleic acid hybridization reactions, the conditions used to achieve a particular level of stringency will vary depending on the nature of the nucleic acids being hybridized. For example, the length, degree of complementarity, nucleotide sequence composition (e.g., GC/AT content), and nucleic acid type (e.g., RNA/DNA) of the hybridizing regions of the nucleic acids can be considered in selecting hybridization conditions. An additional consideration is whether one of the nucleic acids is immobilized, for example, on a filter.

An example of progressively higher stringency conditions is as follows: 2×SSC/0.1% SDS at room temperature (hybridization conditions); 0.2×SSC/0.1% SDS at room temperature (low stringency conditions); 0.2×SSC/0.1% SDS at 42° C. (moderate stringency conditions); and 0.1×SSC at about 68° C. (high stringency conditions). Washing can be carried out using only one of these conditions, e.g., high stringency conditions, or each of the conditions can be used, e.g., for 10-15 minutes each, in the order listed above, repeating any or all of the steps listed. However, as mentioned above, optimal conditions will vary depending on the particular hybridization reaction involved, and can be determined empirically. In general, conditions of high stringency are used for the hybridization of the probe of interest.

Label

The probe of interest can be detectably labeled, for example, with a radioisotope, a fluorescent compound, a bioluminescent compound, a chemiluminescent compound, a metal chelator, or an enzyme. Appropriate labeling with such probes is widely known in the art and can be performed by any conventional method.

### EXAMPLES

Hereinafter, the present invention will be described in further detail with reference to examples. It will be obvious to a person having ordinary skill in the art that these examples are illustrative purposes only and are not to be construed to limit the scope of the present invention.

Measurement of Methylation of SDC2 Biomarker Gene in Tissue of Gastric Polyp Patients

In order to evaluate the usefulness of the SDC2 biomarker gene for early diagnosis of gastric polyp that is a precancerous lesion, genomic DNA was isolated from normal gastric tissues (Biochain; 5 samples) and the paraffin tissues of gastric polyp patients (provided by the Biobank of the Chungnam National University Hospital; 10 low-grade dysplasia samples, and 10 high-grade dysplasia samples). Isolation of genomic DNA from the paraffin tissues was performed using a QIAamp DNA Micro Kit (Qiagen, Germany) according to the manufacturer's instruction.

Measurement of methylation was performed using a quantitative pyrosequencing method. 200 ng of the isolated genomic DNA was treated with bisulfite using an EZ DNA methylation-Gold kit (Zymo Research, USA). When the DNA was treated with bisulfite, unmethylated cytosine was modified to uracil, and methylated cytosine remained without changes. The DNA treated with bisulfite was eluted with 20 μl of sterile distilled water and subjected to pyrosequencing. PCR and sequencing primers for performing pyrosequencing for the SDC2 gene were designed using PSQ assay design program (Qiagen, Germany). The PCR and sequencing primers for measuring the methylation of each gene are shown in Table 1.

TABLE 1
Primers for bisulfite-PCR and pyrosequencing

| | | | SEQ | | Size of |
|---|---|---|---|---|---|
| | | | ID | CpG | amplicon |
| Genes | Primers | Sequences (5′→ 3′)^{a} | NO: | location^{b} | (bp) |
| SDC2 | Forward | GGGAGTGTYGAAATTAATAAGTG | 2 | +460, | 149 |
| | Reverse | Biotin- | 3 | +366, | |
| | | ACCAAAACAAAACRAAACCTCCT | | +473, | |
| | Sequencing | ACCCAAGGAGGAGGAAGYGAG | 4 | +479 | |

^{a}Y = C or T; R = A or G
^{b}distances (nucleotides) from the transcription start site (+1): the positions of CpG regions on the genomic DNA used in the measurement of methylation

20 ng of the genomic DNA treated with bisulfite was amplified by PCR. In the PCR amplification, a PCR reaction solution (20 ng of the genomic DNA treated with bisulfite, 5 μl of 10×PCR buffer (Enzynomics, Korea), 5 units of Taq polymerase (Enzynomics, Korea), 4 μl of 2.5 mM dNTP (Solgent, Korea), and 2 μl (10 pmole/μl) of PCR primers) was used, and the PCR reaction was performed under the following conditions: predenaturation at 95° C. for 5 min, and then 45 cycles of denaturation at 95° C. for 40 sec, annealing at 60° C. for 45 sec and extension at 72° C. for 40 sec, followed by final extension at 72° C. for 5 min. The amplification of the PCR product was confirmed by electrophoresis on 2.0% agarose gel.

The amplified PCR product was treated with PyroGold reagents (Biotage, USA), and then subjected to pyrosequencing using the PSQ96MA system (Biotage, USA) according to the manufacturer's instruction. After the pyrosequencing, the methylation level of the DNA was measured by calculating the methylation index. The methylation index was calculated by determining the average rate of cytosine binding to each CpG island.

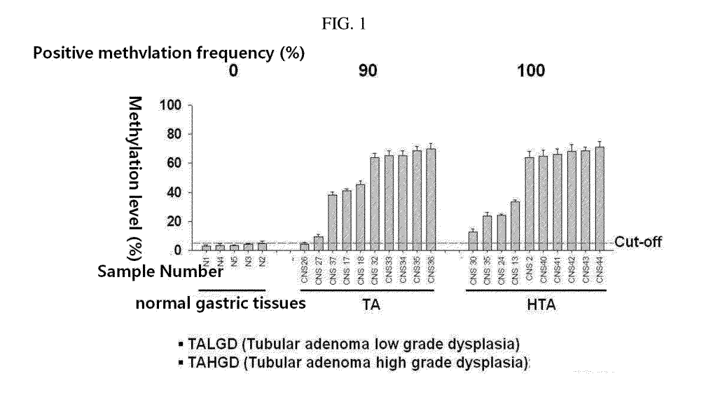

As a result, as can be seen in FIG. 1, the normal gastric tissues showed no methylation, and the low-grade dysplasia showed a methylation frequency of 90.0% (9/10), and the high-grade dysplasia showed a methylation frequency of 100% (10/10).

Such results indicate that the SDC2 biomarker gene showed a high level and frequency of methylation even in gastric polyp that is a precancerous lesion of gastric cancer, suggesting that the SDC2 biomarker gene is useful as a biomarker for diagnosis of gastric polyp and early diagnosis of gastric cancer.

### Example 2

### Measurement of Methylation of Biomarker Gene in Gastric Cancer Cell Line and Gastric Cancer Tissue

In order to examine whether the SDC2 biomarker gene confirmed to be methylated in gastric polyp is also useful as a biomarker for diagnosis of gastric cancer, pyrosequencing was performed in the same manner as described in Example 1.

Genomic DNA was isolated from the gastric cancer cell line AGS (Korean Cell Line Bank (KCLB No. 21739)), and the cancer tissues of 41 gastric cancer patients and normal tissues adjacent to the cancer tissues (provided by the Biobank of the Chungnam National University Hospital) using a QIAamp DNA mini Kit (Qiagen, Germany) according to the manufacturer's instruction.

200 ng of the isolated genomic DNA was treated with bisulfite using an EZ DNA methylation-Gold kit (Zymo Research, USA). When the genomic DNA was treated with bisulfite, unmethylated cytosine was modified to uracil, and methylated cytosine remained without changes. The DNA treated with bisulfite was eluted with 20 μl of sterile distilled water and subjected to pyrosequencing.

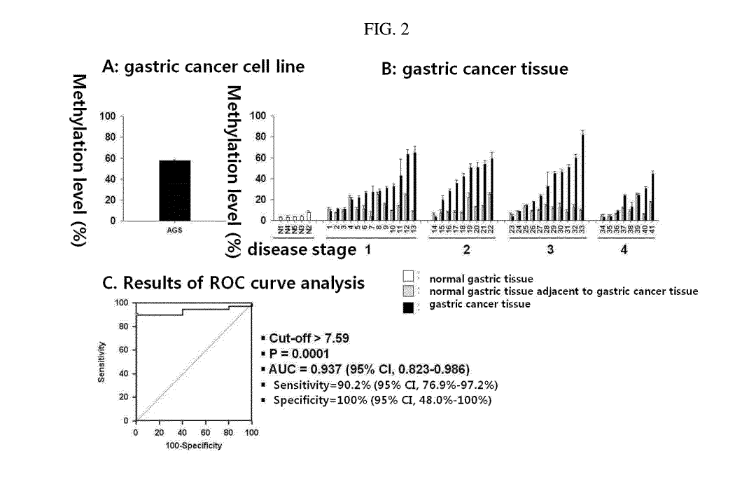

FIG. 2A shows the results of quantitatively measuring the methylation levels of the SDC2 biomarker gene in the gastric cancer cell line by the pyrosequencing method. As can be seen therein, the SDC2 biomarker gene was expressed at a high level in the gastric cancer cell line AGS. This suggests that the SDC2 gene shows a high level of methylation in the gastric cancer cell line, indicating that the SDC2 gene is useful as a biomarker for diagnosis of gastric cancer.

In order to verify this suggestion, an experiment on the verification of methylation of the SDC2 gene in a gastric cancer tissue sample was performed.

To verify the methylation of the SDC2 gene in gastric cancer tissue, the methylation levels of the SDC2 gene in gastric cancer tissues at various disease stages (disease stage 1: 13 persons; disease stage 2: 9 persons; disease stage 3: 11 persons; and disease stage 4: 8 persons) were measured in the same manner as described in Example 1.

As a result, as shown in FIG. 2B, the methylation level of the SDC2 gene in the gastric cancer tissue was significantly higher than those in the gastric tissue of the normal persons and the normal gastric tissue adjacent to the gastric cancer tissue.

In addition, in order to evaluate the sensitivity and specificity of the SDC2 gene for gastric cancer diagnosis, ROC curve analysis was performed. As a result, the gene showed a high sensitivity of 90.2% and a very high specificity of 100% (FIG. 2C). Such results indicate that the SDC2 methylation biomarker gene is useful for gastric cancer diagnosis.

### Example 3

### Measurement of Methylation of SDC2 Biomarker Gene in Sera of Gastric Cancer Patients

In order to examine the usefulness of the SDC2 biomarker gene as a biomarker for gastric cancer diagnosis using serum, the methylation of the SDC2 biomarker gene in the sera of gastric cancer patients was measured by a quantitative methylation-specific real time PCR (qMSP) method.

For this purpose, two PCR primers (IDT, USA) capable of specifically amplifying methylated SDC2 gene treated with bisulfite, and a fluorescent probe (IDT, USA), were designed. To determine the amount and quality of serum DNA treated with bisulfite, ACTB gene was used as an internal control. The sequences of the PCR primers and fluorescent probe used in qMSP are shown in Table 2 below.

TABLE 2
Sequences of primers and fluorescent probe for
qMSP

| | | | Size of |
|---|---|---|---|
| | | | amplified |
| | | SEQ ID | product |
| Gene | Sequences (5′→ 3′) | NO: | (bp) |
| SDC2 | Forward: TAGAAATTAATAAGT | 5 | 121 |
| | GAGAGGGCGT | | |
| | Reverse: GACTCAAACTCGAAA | 6 | |
| | ACTCGAA | | |
| | Fluorescent probe: FAM- | 7 | |
| | AGTAGGCGTAGGAGGAGGAAGCGA- | | |
| | Iowa Black | | |
| ACTB | Forward: TGGTGATGGAGGAGG | 20 | 136 |
| | TTTAGTAAGT | | |
| | Reverse: AACCAATAAAACCTA | 21 | |
| | CTCCTCCCTTAA | | |
| | Fluorescent probe: TET- | | |
| | ACCACCACCCAACACACAATAACA | 22 | |
| | AACACA-Iowa Black | | |

Genomic DNA was isolated from 800 ul of serum using a DynalBeads SILANE viral NA kit (Invitrogen) according to the manufacturer's instruction. The isolated genomic DNA was treated with bisulfite using an EZ DNA methylation-Gold kit (Zymo Research, USA), and then eluted with 20 μl of sterile distilled water and used in qMSP. ⅓ of the volume of the genomic DNA treated with bisulfite used in qMSP. Real-time PCR was performed using a Rotor Gene-Q Real Time PCR system (Qiagen, Germany) with a Rotor Gene Probe Kit (Qiagen, Germany). A final volume of 20 μl was subjected to real-time PCR under the following conditions: for SDC2, 10 min at 95° C., and 50 cycles, each consisting of 10 sec at 95° C., 1 sec at 62° C. and 20 sec at 72° C.; for ACTB, 10 min at 95° C., and 50 cycles, each consisting of 10 sec at 95° C., 60 sec at 58° C.

The methylation level was measured as the Percentage of Methylated Reference (PMR) by a Comparative Cycle Threshold (Ct) method, and the artificially methylated genomic DNA of the gastric cancer cell line AGS (Korean Cell Line Bank (KCLB No. 21739)) was used as a reference. The PMR was calculated using the following equation: PMR=2^{-ΔΔCt}×100; ΔΔCt=[(C_{tSDC2}−C_{tACTB})ₛₐₘₚₗₑ]−[(C_{tSDC2}−C_{tACTB})_{AGS}].

To evaluate the ability of the SDC2 biomarker gene to diagnose gastric cancer in serum, DNA in the sera of 130 normal persons and 40 gastric cancer patients was subjected to qMSP.

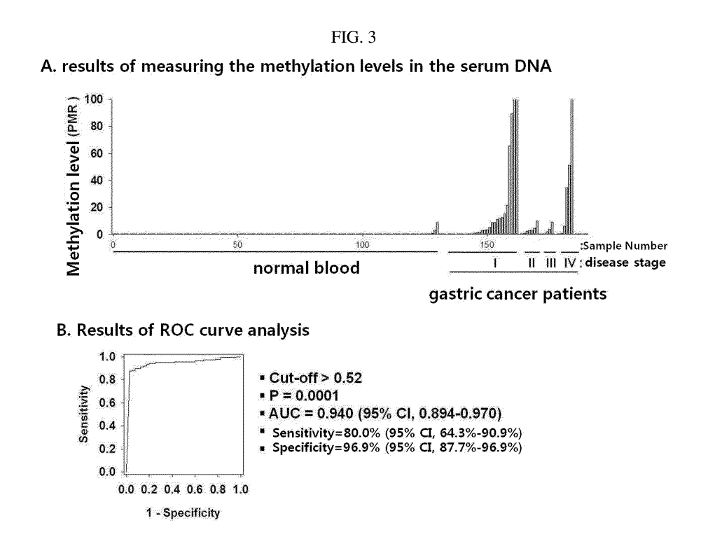

As a result, as can be seen in FIG. 3A, the sera of the normal persons showed little or no methylation of the SDC2 biomarker gene, and the sera of the gastric cancer patients showed methylation of the SDC2 biomarker gene at a high level and frequency. Particularly, the sera at disease stages 1 and 2 of gastric cancer showed a high level and frequency of methylation of the SDC2 biomarker gene.

To measure the sensitivity and specificity of the SDC2 biomarker gene for gastric cancer diagnosis, ROC curve (Receiver Operating Characteristic) analysis was performed using MedCalc program (MEDCALC, Belgium).

As a result, as shown in FIG. 3B, the SDC2 gene showed a sensitivity and specificity of 80.0% and 96.9%, respectively, suggesting that it has a very high ability to diagnose gastric cancer. Particularly, the SDC2 gene showed a sensitivity of 80.0% for early-stage gastric cancer or advanced gastric cancer, suggesting that it is useful for early diagnosis of gastric cancer.

### Example 4

### Measurement of Methylation of SDC2 Biomarker Genes in Other Solid Cancer Tissues

In order to examine whether the SDC2 biomarker gene can be specifically used as a gastric polyp- and gastric cancer-specific diagnostic marker, experiments on other kinds of cancer were performed. In order for the SDC2 biomarker gene to be used as a marker for diagnosis of gastric polyp or gastric cancer, it should not be methylated in various organ tissues of normal persons other than patients and in other solid cancer tissues.

To verify whether the SDC2 biomarker gene satisfies this requirement, genomic DNA was separated from various organic tissues (Biochain) of normal persons other than patients, various solid cancer tissues and normal tissues adjacent to the cancer tissues (provided by the Biobank of the Chungnam National University Hospital) using a QIAamp DNA Mini kit (QIAGEN, USA). 200 ng of the isolated genomic DNA was treated with bisulfite using an EZ DNA methylation-Gold kit (Zymo Research, USA), and then eluted with 20 μl of sterile distilled water and used in pyrosequencing.

20 ng of the genomic DNA treated with bisulfite was amplified by PCR. In the PCR amplification, a PCR reaction solution (20 ng of the genomic DNA treated with bisulfite, 5 μl of 10×PCR buffer (Enzynomics, Korea), 5 units of Taq polymerase (Enzynomics, Korea), 4 μl of 2.5 mM dNTP (Solgent, Korea), and 2 μl (10 pmole/μl) of PCR primers) was used, and the PCR reaction was performed under the following conditions: predenaturation at 95° C. for 5 min, and then 45 cycles of denaturation at 95° C. for 40 sec, annealing at 60° C. for 45 sec and extension at 72° C. for 40 sec, followed by final extension at 72° C. for 5 min. The amplification of the PCR product was confirmed by electrophoresis on 2.0% agarose gel.

The amplified PCR product was treated with PyroGold reagents (Biotage, USA), and then subjected to pyrosequencing using the PSQ96MA system (Biotage, USA). After the pyrosequencing, the methylation level of the SDC2 biomarker gene was measured by calculating the methylation index. The methylation index was calculated by determining the average rate of cytosine binding to each CpG island.

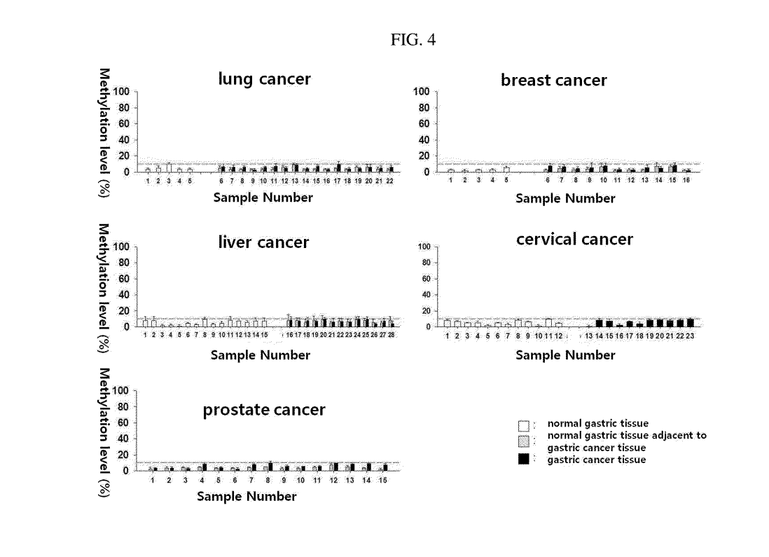

As a result, as can be seen in FIG. 4, the methylation level of the SDC2 biomarker gene was 10% or lower in all of lung cancer tissue, breast cancer tissue, liver cancer tissue, cervical cancer tissue and prostate cancer tissue. This suggests that the SDC2 biomarker gene is methylated specifically in gastric cancer tissue. Such results indicate that the SDC2 biomarker gene can be used as a biomarker not only for gastric cancer-specific diagnosis, but also for diagnosis of bowel cancer.

### Example 5

### Measurement of Methylation of SDC2 Biomarker Gene in Sera of Gastric Polyp Patients

In order to confirm the usefulness of the SDC2 biomarker gene for diagnosis of gastric polyp in serum, the methylation of the SDC2 biomarker gene in the sera of gastric polyp patients was measured by a quantitative methylation-specific real time PCR (qMSP) method. qMSP was performed in the same manner as described in Example 3.

To evaluate the ability of the SDC2 biomarker gene to diagnose gastric polyp in serum, qMSP was performed on DNA in the sera of 12 normal persons determined to be normal by gastroscopy, 5 hyperplastic polyp patients and 16 adenomatous polyp patients. The methylation level was expressed as the cycle threshold (Ct) of the SDC2 gene, and when the Ct value was not formed because methylation was not methylated, the Ct value was expressed as 40.

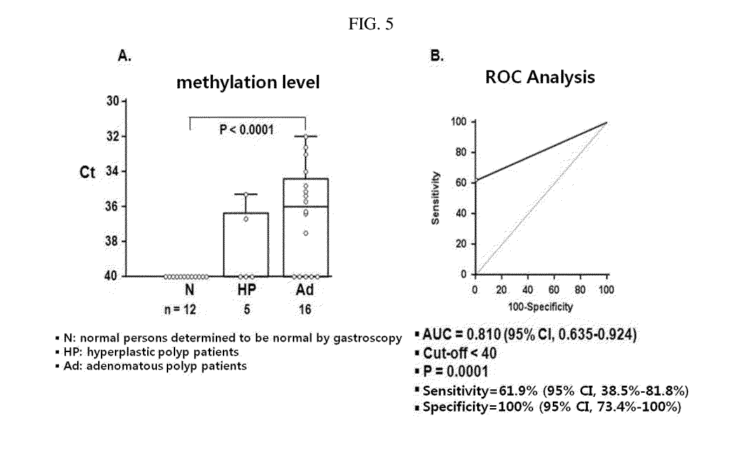

As a result, as can be seen in FIG. 5A, the sera of the 12 normal person showed no methylation of the SDC2 gene, and the sera of the gastric polyp patients showed methylation of the SDC2 gene at a high level and frequency.

To measure the sensitivity and specificity of the SDC2 biomarker gene for gastric polyp diagnosis, ROC curve (Receiver Operating Characteristic) analysis was performed using MedCalc program (MEDCALC, Belgium).

As a result, as can be seen in FIG. 5B, the SDC2 gene showed a sensitivity and specificity of 61.9% (13/21) and 100%, respectively, for the total gastric polyp patients, suggesting that it has an excellent ability to diagnose gastric polyp. In addition, the SDC2 gene showed a sensitivity of 40% (2/5) for hyperplastic polyp and a sensitivity of 68.8% (11/16) for adenomatous polyp. Thus, it was found that the SDC2 gene is highly useful for diagnosis of gastric polyp in blood.

Although the present invention has been described in detail with reference to the specific features, it will be apparent to those skilled in the art that this description is only for a preferred embodiment and does not limit the scope of the present invention. Thus, the substantial scope of the present invention will be defined by the appended claims and equivalents thereof.

### INDUSTRIAL APPLICABILITY

As described above, the present invention has an effect in that the methylation of the CpG island of the gastric polyp- and gastric cancer-specific marker gene can be detected to thereby provide information for diagnosing gastric cancer.

The use of the methylation detection method according to the present invention or the diagnostic composition, kit or nucleic acid chip according to the present invention makes it possible to diagnose gastric cancer at an early transformation stage, thus enabling the early diagnosis of gastric cancer. In addition, the method of the present invention enables gastric cancer to be effectively diagnosed in an accurate and rapid manner compared to conventional methods.

Although the present invention has been described in detail with reference to the specific features, it will be apparent to those skilled in the art that this description is only for a preferred embodiment and does not limit the scope of the present invention. Thus, the substantial scope of the present invention will be defined by the appended claims and equivalents thereof.

1-6. (canceled)

7. A modified nucleic acid for diagnosis of gastric polyp or gastric cancer, derived from a SDC2 (syndecan-2) gene fragment set forth in SEQ ID NO: 1, in which the modified nucleic acid is obtained by modifying the SDC2 gene fragment so that at least one cytosine residue in the SDC2 gene fragment is modified to uracil.

8. The modified nucleic acid of claim 7, the modified nucleic acid comprises a sequence set forth in SEQ ID NO: 23.

9-12. (canceled)

13. A method for detecting gastric cancer, comprising the steps of:
(a) isolating DNA from a clinical sample;
(b) measuring the methylation of the CpG island of modified nucleic acid for diagnosis of gastric cancer, derived from a SDC2 (syndecan-2) gene fragment set forth in SEQ ID NO: 1, in which the modified nucleic acid is obtained by modifying the SDC2 gene fragment so that at least one cytosine residue in the SDC2 gene fragment is modified to uracil, which is a gastric cancer biomarker, in the isolated DNA to detect gastric cancer; and
(c) detecting increased CpG methylation of the modified nucleic acid derived from a SDC2 (syndecan-2) gene fragment set forth in SEQ ID NO: 1, relative to that of a control, to have a gastric cancer.

14. A method for detecting gastric polyp, comprising the steps of:
(a) isolating DNA from a clinical sample;
(b) measuring the methylation of the CpG island of modified nucleic acid for diagnosis of gastric polyp, derived from a SDC2 (syndecan-2) gene fragment set forth in SEQ ID NO: 1, in which the modified nucleic acid is obtained by modifying the SDC2 gene fragment so that at least one cytosine residue in the SDC2 gene fragment is modified to uracil, which is a gastric polyp biomarker, in the isolated DNA; and
(c) detecting increased CpG methylation of SDC2 (syndecan-2) gene relative to that of a control to have a gastric polyp.

15. The method of claim 13 or 14, wherein the modified nucleic acid derived from a SDC2 (syndecan-2) gene fragment set forth in SEQ ID NO: 1 is obtained by treating SDC2 gene with a reagent that modifies a methylated DNA and a non-methylated DNA differently, and then the methylation of the treated gene is measured.

16. The method of claim 15, wherein the reagent is bisulfite.

17. The method of claim 13 or 14, wherein step (b) is performed by measuring the methylation of any one of the promoter, 5′-untranslated region (UTR), intron and exon regions of the gene.

18. (canceled)

19. The method of claim 13 or 14, wherein the modified nucleic acid has a sequence set forth in SEQ ID NO: 23.

20. The method of claim 13 or 14, wherein step (b) is performed by a method selected from the group consisting of PCR, methylation-specific PCR, real-time methylation-specific PCR, PCR using a methylation DNA-specific binding protein, PCR using a methylation DNA-specific binding antibody, quantitative PCR, DNA chip-based assay, sequencing, sequencing-by-synthesis, and sequencing-by-ligation technique.

21. The method of claim 13 or 14, wherein the clinical sample is selected from the group consisting of a tissue, cell, blood, blood plasma, feces, serum, and urine from a patient suspected of cancer or a subject to be diagnosed.

22. A nucleic acid chip for diagnosing gastric cancer, which comprises immobilized thereon a probe capable of hybridizing to a fragment comprising the CpG island of modified nucleic acid, derived from a SDC2 (syndecan-2) gene fragment set forth in SEQ ID NO: 1, in which the modified nucleic acid is obtained by modifying the SDC2 gene fragment so that at least one cytosine residue in the SDC2 gene fragment is modified to uracil, under a strict condition.

23. A nucleic acid chip for diagnosing gastric polyp, which comprises immobilized thereon a probe capable of hybridizing to a fragment comprising the CpG island of modified nucleic acid, derived from a SDC2 (syndecan-2) gene fragment set forth in SEQ ID NO: 1, in which the modified nucleic acid is obtained by modifying the SDC2 gene fragment so that at least one cytosine residue in the SDC2 gene fragment is modified to uracil, under a strict condition.

24. (canceled)

25. The nucleic acid chip of claim 22 or 23, wherein the probe is selected from the group consisting of the sequences set forth in SEQ ID NOS: 14 to 19.

26. The method of claim 13 or 14, wherein step (b) is performed by using primer(s) for amplifying a methylated CpG island of modified nucleic acid, derived from a SDC2 (syndecan-2) gene fragment set forth in SEQ ID NO: 1 or a probe capable of hybridizing to a fragment comprising the CpG island of modified nucleic acid, derived from a SDC2 (syndecan-2) gene fragment set forth in SEQ ID NO: 1 under a strict condition.

27. The method of claim 26, wherein the primer(s) is selected from the group consisting of the sequences set forth in SEQ ID NOS: 2, 3, 5 and 6.

28. The method of claim 27, wherein the primer(s) further comprise a sequencing primer set forth in SEQ ID NO: 4.

29. The method of claim 26, wherein the probe is selected from the group consisting of the sequences set forth in SEQ ID NOS: 14 to 19.

30. The method of claim 26, further comprising a methylation-specific binding protein or a methylation-specific binding antibody which is capable of binding to the methylated CpG island.


SEQUENCE LISTING (23 sequences)

SEQ ID NO: 1 - DNA, 2000 bp, Homo sapiens
ttgggggtgcggaaggatttgggagaatgggaaacactctcactatatatttattacattaattatcttctcttttaaaatgcaattttcatgaactggcgatttatgaacacttcacattgcttgaaagcatcttacactttttttttccctcaactcacaaagcagtttctttctactggtcgaattctcaaggcagaaaagctacatacgtctctcgtttcttcactaattgttctctagaaaagggaaagtgaagaagggaaagagaaaagacaacggggaagaaaagagcatagaggagagaggaaaagtggggagagaaaggaagaaaaggactgagaaaacgcaggagccctggcttgccggtgagcagagccggcgcagccacagcgcggagccgcggcgcccactggtcctcggagctgccaatcggcgtgtaatcctgtaggaatttctcccgggtttatctgggagtcacactgccgcctcctctccccagtcgcccaggggagcccggagaagcaggctcaggagggagggagccagaggaaaagaagaggaggagaaggaggaggacccggggagggaggcgcggcgcgggaggaggaggggcgcagccgcggagccagtggccccgcttggacgcgctgctctccagatacccccggagctccagccgcgcggatcgcgcgctcccgccgctctgcccctaaacttctgccgtagctccctttcaagccagcgaatttattccttaaaaccagaaactgaacctcggcacgggaaaggagtccgcggaggagcaaaaccacagcagagcaagaagagcttcagagagcagccttcccggagcaccaactccgtgtcgggagtgcagaaaccaacaagtgagagggcgccgcgttcccggggcgcagctgcgggcggcgggagcaggcgcaggaggaggaagcgagcgcccccgagccccgagcccgagtccccgagcctgagccgcaatcgctgcggtactctgctccggattcgtgtgcgcgggctgcgccgagcgctgggcaggaggcttcgttttgccctggttgcaagcagcggctgggagcagccggtccctggggaatatgcggcgcgcgtggatcctgctcaccttgggcttggtggcctgcgtgtcggcggagtcggtgagtgggccaggcggaggatgcgcgcgccgtttagggtgtttgaagctacgagaggagcccgcagggaataggggagcgccacctggggaacccccagtccccaagtatacaccggagatccgctgggacaaatgcgctcgtccggtcaccctttccccctcttcccttcctcagaaaagcgctgctcgctggcgttaccccgcggtccgcgggaatgggggcaccgagaattgcggtttggtctagccgcagaggcccctgaagtcactcccaacttcttcgccctcggcgggtcttgctgcgtggtctgggaaggacggaggggaaagggtggcaggaggggggagcctgggtcgggcccgcgagggaacggctccactccgcgcgctcctcgagaccagggatgacctggaaacttcggggtcccttcctccgcacaccatcccccccgcgccagctttcctgtttgactgcatgcaagttctggggagatgggggccagatttaagagacccgcgagtgtccagagagaaaagtttgcaaaagttcttttgtttgatgctccctgcggctagggcgaggtaaccgacactacgtggaatcgcagtaggcgatccctcaaggggatactgggggaggcacggaacgcgtccgaaaatgctgggacgccggccactggattcccagtcctgcggcgaccccctcctcgttgaggggtggaggttgcaccgcggggcgtcagggacgggaggacattttcataggagttacacgggagtgccgcaagcagggcgaggcggggtacgtgtgacacggcgc

SEQ ID NO: 2 - DNA, 23 bp, Artificial Sequence
  Forward primer of SDC2 gene
gggagtgtygaaattaataagtg

SEQ ID NO: 3 - DNA, 28 bp, Artificial Sequence
  Backward primer of SDC2 gene
accaaaacaaaacraaacctcctaccca

SEQ ID NO: 4 - DNA, 16 bp, Artificial Sequence
  Sequencing primer of SDC2 gene
aggaggaggaagygag

SEQ ID NO: 5 - DNA, 25 bp, Artificial Sequence
  Forward primer of SDC2 gene
tagaaattaataagtgagagggcgt

SEQ ID NO: 6 - DNA, 22 bp, Artificial Sequence
  Backward primer of SDC2 gene
gactcaaactcgaaaactcgaa

SEQ ID NO: 7 - DNA, 24 bp, Artificial Sequence
  Fluorescene probe of SDC2 gene
agtaggcgtaggaggaggaagcga

SEQ ID NO: 8 - DNA, 30 bp, Artificial Sequence
  Probe for SDC2 gene
cggagctgccaatcggcgtgtaatcctgta

SEQ ID NO: 9 - DNA, 50 bp, Artificial Sequence
  Probe for SDC2 gene
ctgccgtagctccctttcaagccagcgaatttattccttaaaaccagaaa

SEQ ID NO: 10 - DNA, 50 bp, Artificial Sequence
  Probe for SDC2 gene
gcacgggaaaggagtccgcggaggagcaaaaccacagcagagcaagaaga

SEQ ID NO: 11 - DNA, 60 bp, Artificial Sequence
  Probe for SDC2 gene
gcagccttcccggagcaccaactccgtgtcgggagtgcagaaaccaacaagtgagagggc

SEQ ID NO: 12 - DNA, 50 bp, Artificial Sequence
  Probe for SDC2 gene
cccgagcccgagtccccgagcctgagccgcaatcgctgcggtactctgct

SEQ ID NO: 13 - DNA, 40 bp, Artificial Sequence
  Probe for SDC2 gene
cttggtggcctgcgtgtcggcggagtcggtgagtgggcca

SEQ ID NO: 14 - DNA, 30 bp, Artificial Sequence
  probe
cggagttgttaatcggcgtgtaattttgta

SEQ ID NO: 15 - DNA, 50 bp, Artificial Sequence
  probe
ttgtcgtagtttttttttaagttagcgaatttattttttaaaattagaaa

SEQ ID NO: 16 - DNA, 50 bp, Artificial Sequence
  probe
gtacgggaaaggagttcgcggaggagtaaaattatagtagagtaagaaga

SEQ ID NO: 17 - DNA, 60 bp, Artificial Sequence
  probe
gtagttttttcggagtattaatttcgtgtcgggagtgtagaaattaataagtgagagggt

SEQ ID NO: 18 - DNA, 50 bp, Artificial Sequence
  probe
ttcgagttcgagttttcgagtttgagtcgtaatcgttgcggtattttgtt

SEQ ID NO: 19 - DNA, 40 bp, Artificial Sequence
  probe
tttggtggtttgcgtgtcggcggagtcggtgagtgggtta

SEQ ID NO: 20 - DNA, 25 bp, Artificial Sequence
  Forward primer of ACTB gene
tggtgatggaggaggtttagtaagt

SEQ ID NO: 21 - DNA, 27 bp, Artificial Sequence
  Backward primer of ACTB gene
aaccaataaaacctactcctcccttaa

SEQ ID NO: 22 - DNA, 30 bp, Artificial Sequence
  Fluorescence probe of ACTB gene
accaccacccaacacacaataacaaacaca

SEQ ID NO: 23 - DNA, 2000 bp, Artificial Sequence
  Bisulfite treated promoter, 5′ untranslated region and 1st intron of SDC2 gene
ttgggggtgcggaaggatttgggagaatgggaaatatttttattatatatttattatattaattatttttttttttaaaatgtaatttttatgaattggcgatttatgaatattttatattgtttgaaagtattttatatttttttttttttttaatttataaagtagtttttttttattggtcgaatttttaaggtagaaaagttatatacgtttttcgtttttttattaattgttttttagaaaagggaaagtgaagaagggaaagagaaaagataacggggaagaaaagagtatagaggagagaggaaaagtggggagagaaaggaagaaaaggattgagaaaacgtaggagttttggtttgtcggtgagtagagtcggcgtagttatagcgcggagtcgcggcgtttattggttttcggagttgttaatcggcgtgtaattttgtaggaatttttttcgggtttatttgggagttatattgtcgttttttttttttagtcgtttaggggagttcggagaagtaggtttaggagggagggagttagaggaaaagaagaggaggagaaggaggaggattcggggagggaggcgcggcgcgggaggaggaggggcgtagtcgcggagttagtggtttcgtttggacgcgttgttttttagatattttcggagttttagtcgcgcggatcgcgcgttttcgtcgttttgtttttaaatttttgtcgtagtttttttttaagttagcgaatttattttttaaaattagaaattgaatttcggtacgggaaaggagttcgcggaggagtaaaattatagtagagtaagaagagttttagagagtagttttttcggagtattaatttcgtgtcgggagtgtagaaattaataagtgagagggcgtcgcgttttcggggcgtagttgcgggcggcgggagtaggcgtaggaggaggaagcgagcgttttcgagtttcgagttcgagttttcgagtttgagtcgtaatcgttgcggtattttgtttcggattcgtgtgcgcgggttgcgtcgagcgttgggtaggaggtttcgttttgttttggttgtaagtagcggttgggagtagtcggtttttggggaatatgcggcgcgcgtggattttgtttattttgggtttggtggtttgcgtgtcggcggagtcggtgagtgggttaggcggaggatgcgcgcgtcgtttagggtgtttgaagttacgagaggagttcgtagggaataggggagcgttatttggggaatttttagtttttaagtatatatcggagattcgttgggataaatgcgttcgttcggttattttttttttttttttttttttttagaaaagcgttgttcgttggcgttatttcgcggttcgcgggaatgggggtatcgagaattgcggtttggtttagtcgtagaggtttttgaagttatttttaattttttcgttttcggcgggttttgttgcgtggtttgggaaggacggaggggaaagggtggtaggaggggggagtttgggtcgggttcgcgagggaacggttttatttcgcgcgtttttcgagattagggatgatttggaaatttcggggttttttttttcgtatattatttttttcgcgttagtttttttgtttgattgtatgtaagttttggggagatgggggttagatttaagagattcgcgagtgtttagagagaaaagtttgtaaaagtttttttgtttgatgttttttgcggttagggcgaggtaatcgatattacgtggaatcgtagtaggcgattttttaaggggatattgggggaggtacggaacgcgttcgaaaatgttgggacgtcggttattggatttttagttttgcggcgatttttttttcgttgaggggtggaggttgtatcgcggggcgttagggacgggaggatatttttataggagttatacgggagtgtcgtaagtagggcgaggcggggtacgtgtgatacggcgt